(19) United States
(12) Patent Application Publication
(10) Pub. No.: US 2004/0009598 A1
(43) Pub. Date: Jan. 15, 2004

(54) Use of bioactive glass compositions to stimulate osteoblast production

(76) Inventors: Larry L Hench, London (GB); Julia M Polak, London (GB); Lee D.k. Buttery, London (GB); Ioannis D Xynos, Nafplion (GR); Jason Maroothynaden, London (GB)

Correspondence Address: BURNS DOANE SWECKER & MATHIS L L P, POST OFFICE BOX 1404, ALEXANDRIA, VA, 22313-1404, US

(21) Appl. No.: 10/332,731

(22) PCT Filed: Jul. 11, 2001
(86) PCT No.: PCT/US01/21801

(51) Int. Cl. C12N 5/00; C12N 5/02

(52) U.S. Cl. 435/375; 435/395

(57) ABSTRACT

Compositions comprising bioactive glass compositions or extracts thereof which include ions in an appropriate concentration and ratio that they enhance osteoblast production, and methods of preparation and use thereof, are disclosed. The compositions can be included in implantable devices that are capable of inducing tissue formation in autogeneic, allogeneic and xenogeneic implants, for example as coatings and/or matrix materials. Examples of such devices include prosthetic implants, sutures, stents, screws, plates, tubes, and the like. Aqueous extracts of the bioactive glass compositions, which extracts are capable of stimulating osteoblast production, are also disclosed. The compositions can be used, for example, to induce local tissue formation from a progenitor cell in a mammal, for accelerating allograft repair in a mammal, for promoting in vivo integration of an implantable prosthetic device to enhance the bond strength between the prosthesis and the existing target tissue at the joining site, and for treating tissue degenerative conditions.

### FIELD OF THE INVENTION

The present invention is generally in the area of methods for repair and reconstruction of bone, cartilage and enhancement of healing of other tissues.

### BACKGROUND OF THE INVENTION

Bone is a dense network of collagen protein fibers arranged in layers with crystals of hydrated and carbonated calcium phosphate between the fibers, where about 25% of the weight is calcium. Living cells called osteocytes are arranged in lacunae throughout the bone. Very small blood vessels extend throughout the bone and supply the osteocytes with oxygen and nutrients. The natural process for repairing bone defects involves having osteoclasts remove damaged bone, and then having osteoblast cells lay down new bone. The osteoblasts repeatedly form layers, each consisting of a network of collagen fibers, which produce enzymes resulting in calcium and phosphorus deposition as crystalline hydroxy carbonate apatite until the defect is repaired.

Relatively small bone defects can be repaired using bone cements, pins, screws and other devices for mechanical stabilization. Relatively large defects typically require that the missing bone be replaced with a biocompatible material that provides support and which can be immobilized. Bone grafts are often necessary when bone fails to repair itself or when bone loss occurs through fracture or tumor. Bone grafts have to provide mechanical stability and be a source of osteogenesis. Bone grafting is described, for example, in Friedlaender, G. E., “Current Concepts Review: Bone Grafts,” Journal of Bone and Joint Surgery, 69A(5), 786-790 (1987). Osteoinduction and osteoconduction are two mechanisms by which a graft may stimulate the growth of new bone. In osteoinduction, inductive signals lead to the phenotypic conversion of progenitor cells to bone cells. In osteoconduction, the implant provides a scaffold for bony ingrowth. The bone remodeling cycle is a continuous event involving the resorption of pre-existing bone by osteoclasts and the formation of new bone by the work of osteoblasts.

Bony defects are commonly treated using grafts of organic and synthetic construction, typically autografts, allografts, and xenografts. An autograft is tissue transplanted from one site to another in the patient. The benefits of using the patient's tissue are that the graft will not evoke a strong immune response and that the material may or may not be vascularized, which allows for speedy incorporation. However, using an autograft requires a second surgery, which increases the risk of infection and introduces additional weakness at the harvest site. Further, bone available for grafting may be removed from a limited number of sites, for example the fibula, ribs and iliac crest. An allograft is tissue taken from a different organism of the same species, and a xenograft from an organism of a different species. The latter types of tissue are readily available in larger quantities than autografts, but genetic differences between the donor and recipient may lead to graft rejection.

Synthetic materials have also been used, for example titanium and steel alloys, particularly those having a porous structure to allow cellular ingrowth to stabilize the implant, bone cements, alone or mixed with cells, sterilized bone, and polymeric or polymeric/hydroxyapatite implants. All have advantages and disadvantages, yet none provides a perfect replacement for the missing bone.

Large defects are particularly difficult to treat. One approach involves using tissue engineering to stimulate production of osteoblasts or bone tissue. It would be advantageous to provide new compositions and methods for stimulating osteoblast production. The present invention provides such compositions and methods.

### SUMMARY OF THE INVETION

Compositions comprising bioactive glass compositions or extracts thereof, which include ions in an appropriate concentration and ratio that they enhance osteoblast production, and methods of preparation and use thereof, are disclosed.

The compositions can be included in implantable devices that are capable of inducing tissue formation at the implant site, for example as coatings and/or matrix materials. Examples of such devices include prosthetic implants, sutures, stents, screws, plates, tubes, and the like.

Aqueous extracts of the bioactive glass compositions, which extracts are capable of stimulating osteoblast production, are also disclosed. Such extracts can be formed by placing bioactive glass in an aqueous solution, allowing the glass to dissolve over a suitable period of time, for example one day or more, and filtering out the undissolved glass particles. The solvent can also be evaporated to provide a solid material with osteoblast-stimulating properties. Alternatively, the solutions can be prepared by mixing the correct ions in an appropriate concentration rather than by extraction from bioactive glass.

The compositions can be used, for example, to induce local tissue formation from a progenitor cell in a mammal, for accelerating allograft repair in a mammal, for promoting in vivo integration of an implantable prosthetic device to enhance the bond strength between the prosthesis and the existing target tissue at the joining site, and for treating tissue degenerative conditions in a mammal.

### DETAILED DESCRIPTION OF THE PREFERRED EMBODIMENTS

Biocompatible compositions and methods for enhancing osteoblast production using the compositions are disclosed. The compositions include an osteoblast-stimulating bioactive glass or extract thereof with a ratio and/or concentration of ions that stimulates osteoblast proliferation, differentiation and/or finction. A major function of osteoblasts is the formation of new bone or other tissues, such as those involved in the process of membranous or endochondral bone formation. While not wishing to be bound to a particular theory, it is believed that exposure of human osteoblast cells to the ions results in up-regulation of certain cytokines, proteoglycans and/or other proteins such as growth factors that are implicated in the growth, differentiation and control of bone formation in humans. Genes whose expression is enhanced by exposure to the bioactive glass solutions include c-jun and c-myc genes, which are implicated in the early events of cell proliferation and differentiation. In some cases, up-regulation is observed even after 48 hours post-exposure.

In general, the genes shown to be upregulated by exposure to the bioactive glass or bioactive glass extract compositions of the invention are involved in:

a) signaling to produce proteins responsible for cell binding,

b) up-regulation of the osteoblast cell cycle, thus stimulating new cell development,

c) enhancing collagen synthesis, and

d) controlling apoptosis, thereby increasing the rate of the cell cycle.

Exposure to the compositions also increases expression of insulin-like growth factor-II (IGF-II), an abundant mitogenic molecule found in bone which stimulates chondrocyte activity and osteoblast proliferation and differentiation. It is believed to appear earlier in the bone regeneration cycle than bone morphogenic proteins (BMPs). The term “biocompatible” refers to a material that does not elicit detrimental effects associated with the body's various protective systems, such as cell and humoral-associated immune responses, e.g., inflammatory responses and foreign body fibrotic responses. The term biocompatible also implies that no specific undesirable cytotoxic or systemic effects are caused by the material when it is implanted into the patient.

The terms “morphogenic activity,” “inducing activity” and “tissue inductive activity” alternatively refer to the ability of an agent to stimulate a target cell to undergo one or more cell divisions (proliferation) that can optionally lead to cell differentiation. Such target cells are referred to generically herein as progenitor cells. Cell proliferation is typically characterized by changes in cell cycle regulation and can be detected by a number of means which include measuring DNA synthesis or cellular growth. Early stages of cell differentiation are typically characterized by changes in gene expression patterns relative to those of the progenitor cell, which can be indicative of a commitment towards a particular cell fate or cell type. Later stages of cell differentiation can be characterized by changes in gene expression patterns, cell physiology and morphology. Any reproducible change in gene expression, cell physiology or morphology can be used to assess the initiation and extent of cell differentiation induced by the compositions described herein.

Observed Effect in Cell Culture

The bioactive glass or bioactive glass extract compositions described herein, when added to cells in culture, were observed to have the following effects:

The population of cells in primary human osteoblast cultures that are capable of dividing and proliferating increased;

The population of cells in primary human osteoblast cultures that are not dividing, proliferating, or differentiating, or producing extra-cellular matrices undergo rapid apoptosis;

The cells in primary human osteoblast cultures that are capable of dividing and proliferating showed a more rapid differentiation from an osteoblast precursor towards a mature phenotype characteristic of osteocytes;

Mineralized bone nodules were rapidly formed in primary human osteoblast cultures;

The cells in mouse embryonic cell cultures underwent rapid selection and differentiation into cells of the osteoblast lineage;

Rapid mineralization of the femora was observed in mouse fetal femoras in culture, even under micro-gravity conditions where mineralization does not occur in the absence of the compositions;

Enhanced mineralization of the metatarsals was observed in mouse fetal metatarsals in culture, even under simulated hyper-gravity conditions; and

A series of genes which influence growth and formation of new bone was up-regulated in human osteoblast cultures. These genes include IGF-II, IGFBP3, MMP2, MMP14, TIMPI, TIMP2, procollagen a2, Decorin, c-jun, c-myc, calcium proteinase (calpain) and DAD 1 (defender against cell death). The most significant up-regulation was observed with IGF-II, MMP2, MMP 14, TIMP1, calpain and DAD 1.

The effect varied depending on the concentration of the ions in solution when aqueous extracts of bioactive glass were used. For example, for extracts derived from 45S5 Bioglass, when the concentration was about 10 g/l, the effect was optimized, and either below 2 g/l or above 40 g/l in culture, was not significantly observed. For extracts derived from other bioactive glass compositions, the concentrations will be expected to be different.

An effective amount of bioactive glass or bioactive glass extract for stimulation of osteoblast production, or osteoblast proliferation, differentiation, function or a combination thereof, will be an amount which will provide at least one of the above-listed effects.

I. Bioactive Glass Compositions

The compositions include osteoblast-stimulating bioactive glass, preferably in the form of fibers, particles, preferably non-interlinked particles, extracts derived from the bioactive glass, and sols, gels or solids derived from the extracts. The compositions can optionally include other therapeutic agents.

As used herein, the terms “bioactive glass” or “biologically active glass” mean an inorganic glass material having an oxide of silicon as its major component and which is capable of bonding with growing tissue when reacted with physiological fluids. The term “osteoblast-stimulating” refers to bioactive glasses and aqueous extracts thereof with particular ratios and/or concentrations of ions which stimulate osteoblast proliferation, differentiation and/or function.

Bioactive glasses are well known to those skilled in the art, and are disclosed, for example, in An Introduction to Bioceramics, L. Hench and J. Wilson, eds. World Scientific, New Jersey (1993).

The glass includes a composition by approximate weight percent of between about 42 and 52% by weight of silicon dioxide (SiO₂), between about 15 and 25% by weight of sodium oxide (Na₂O), between about 15 and 25% by weight calcium oxide (CaO), and between about 1 and 9% by weight phosphorus oxide (P₂O₅), when the glass is melt-derived. The glass includes between about 55 and 80% by weight of silicon dioxide (SiO₂), between about 0 and 9% by weight of sodium oxide (Na₂O), between about 10 and 40% by weight calcium oxide (CaO), and between about 3 and 8% by weight phosphorus oxide (P₂O₅), when the glass is sol gel-derived. The oxides can be present as solid solutions or mixed oxides, or as mixtures of oxides. The currently most preferred glass is 45S5 bioglass, which has a composition by weight percentage of approximately 45% SiO₂, 24.5% CaO, 24.5% Na₂O and 6% P₂O₅.

CaF₂, B₂O₃, A1₂O₃, MgO, Ag₂O, ZnO and K₂O can be included in the composition in addition to silicon, sodium, phosphorus and calcium oxides. The preferred range for B₂O₃ is between 0 and 10% by weight. The preferred range for K₂O is between 0 and 8% by weight. The preferred range for MgO is between 0 and 5% by weight. The preferred range for Al₂O₃ is between 0 and 1.5% by weight. The preferred range for CaF₂ is between 0 and 12.5 % by weight. The preferred range for Ag₂O and ZnO is between 0 and 2% by weight.

Particulate, non-interlinked bioactive glass is preferred. That is, the glass is in the form of small, discrete particles, rather than a fused matrix of particles or a mesh or fabric (woven or non-woven) of glass fibers. Note that under some conditions the discrete particles of the present invention can tend to cling together because of electrostatic or other forces but are still considered to be non-interlinked. Useful ranges of particle sizes are less than about 1200 microns, typically between 1 and 1000 microns. For direct implantation, the particle size range depends on the intended application. In one embodiment, the size range of the particles is about 100 to about 800 microns. In a preferred aspect of the invention, the size range of the particles is about 300 to about 700 microns. To produce extracts, the particle size is preferably less than about 90 microns; more preferably, less than about 20 microns; even more preferably, less than about 5 microns, and ideally, less than about 3 microns, as measured by SEM or laser light scattering techniques.

Highly porous bioactive glass can also be used, particularly in tissue engineering applications where the high porosity can be useful in matrix materials for cell culture. Highly porous bioactive glass has a relatively fast degradation rate and high surface area, in comparison to non-porous bioactive glass compositions. When highly porous bioactive glass is used in place or in addition to small particles of bioactive glass, the pore size is between about 0 and 500 μm, preferably between about 50 and 500 μm, more preferably between 100 and 400 μm. The degree of porosity of the glass is between about 0 and 85%, preferably between about 30 and 80%, and more preferably between about 40 and 60 %. Porous bioactive glass can be prepared, for example, by incorporating a leachable substance into the bioactive glass composition, and leaching the substance out of the glass. Suitable leachable substances are well known to those of skill in the art and include, for example, sodium chloride and other water-soluble salts. The particle size of the leachable substance is roughly the size of the resulting pore. The relative amount and size of the leachable substance gives rise to the degree of porosity. Also, as described herein, porosity can be achieved using sintering and/or by controlling the treatment cycle of glass gels to control the pores and interpores of the material.

The glass composition can be prepared in several ways, to provide melt-derived glass, sol-gel derived glass, and sintered glass particles. The sintered particles can be in sol-gel derived, or pre-reacted melt derived form. Sol-gel derived glass is generally prepared by synthesizing an inorganic network by mixing metal alkoxides in solution, followed by hydrolysis, gelation, and low temperature (around 200-900° C.) firing to produce a glass. Sol-gel derived glasses produced this way are known to have an initial high specific surface area compared with either melt-derived glass or porous melt-derived glass. Melt derived glass is generally prepared by mixing grains of oxides or carbonates, melting and homogenizing the mixtures at high temperatures, typically between about 1250 and 1400° C. The molten glass can be fritted and milled to produce a small particulate material.

The glass composition is preferably melt-derived. In each preparation, it is preferred to use reagent grade glass and/or chemicals, especially since the glass and/or chemicals are used to prepare materials which ultimately can be administered to a patient.

A. Melt Derived Glass

A melt-derived glass composition can be prepared, for example, by preparing an admixture of the individual metal oxides and other components used to prepare the glass composition, blending the admixture, melting the admixture, and cooling the mixture. The melting temperature is determined in large part by the glass composition, and ranges, for example, from about 900-1500° C., preferably between about 1250 and 1450° C. The melt is preferably mixed, for example, by oxygen bubbling, to ensure a thorough homogenation of the individual components.

The mixture can be cooled, for example by casting the molten admixture into a suitable liquid such as deionized water, to produce a glass frit. Porosity can be introduced by grinding the glass into a powder, admixing the powder with a foaming agent, and hot pressing the mixture under vacuum and elevated temperature. The particle size of the glass powder is between about 2 and 70 μm, the vacuum is preferably less than 50 MPa, and the hot pressing is preferably performed at a temperature above 400° C., preferably between about 400 and 500° C. Suitable foaming agents include compounds which evolve carbon dioxide and/or water at elevated temperatures, for example metal hydroxides, metal carbonates, and peroxides such as hydrogen peroxide. Preferred metal carbonates are sodium bicarbonate, sodium carbonate and calcium carbonate. The foaming agents are preferably added in a range of between about 1-5, more preferably 2-3 percent by weight of the glass powder. The preparation of melt-derived porous glass is described, for example, in U.S. Pat. No. 5,648,301 to Ducheyne and El Ghannam.

B. Sintered Glass Particles

Glass can be sintered using known methodology. In one embodiment, an aqueous slurry of the glass powder and a foaming agent with a suitable binder, such as polyvinyl alcohol, is formed. The slurry is then poured into a mold, allowed to dry, and sintered at high temperatures. These temperature can range, depending on the glass composition and foaming agent used, between about 450 and 1000° C., more preferably between about 550 and 800° C.

C. Leaching of the Porous Material

To aid in preparing glass compositions with high porosity, the glass composition can include a material which can be preferably leached out of the glass composition, and in doing so, provide the composition with high porosity. For example, minute particles of a material capable of being dissolved in a suitable solvent, acid or base can be mixed with or melted into the glass, and subsequently leached out. The resulting voids have roughly the same size as the particle that was leached out. In the case of a material which is part of a melt-derived glass composition, the size of the pores and degree of porosity depends on the amount of added material relative to the amount of glass. For example, if the leached material constituted about 80% of the glass, then the glass would be approximately 80% porous when the material was leached out. When leaching the glass composition, care should be taken not to leach out those components which add to the bioactivity of the glass, i.e., the calcium, silica and phosphorus oxides.

II. Solutions Derived from Bioactive Glass

Osteoblast-stimulating compositions derived from aqueous or other extracts of bioactive glass, and/or solutions including the same ions at the same concentration ranges can be used in the methods described herein. The extracts can be formed by placing an osteoblast-stimulating bioactive glass in an aqueous solution, allowing the glass to dissolve over a suitable period of time, and filtering out the un-dissolved glass particles. The solvent can be evaporated to provide a sol, gel or solid material with osteoblast-stimulating properties. The compositions can be used in situations where osteoblast production is desired, for example solutions used for cell culture, and buffer solutions.

The extract may be incorporated into hydrogels or other aqueous based biocompatible carriers for delivery to specific sites in the body. Those of skill in the art will appreciate that the molecular weight and/or water content of polymers or other materials utilized as carriers may be used to control the rate of release of the ionic bioactive glass extracts.

The concentration of ions in aqueous osteoblast-enhancing solutions is as follows:

| | |
|---|---|
| Si - | 1 ppm to 100 ppm |
| Ca | 10 ppm to 150 ppm |
| P | 5 ppm to 50 ppm. |

Typically, the osteoblast-enhancing solutions will also contain sodium ions. The amount will depend on the environment in which the solution is used and the amount of time of reaction of the initial glass composition.

The preferred range of ions is:

| | |
|---|---|
| Si | 3 ppm to 40 ppm |
| Ca | 60 ppm to 100 ppm |
| P | 10 ppm to 40 ppm. |

Without being bound to a particular theory, it is believed that there is a complex relationship between the type of ion being released from the glass, the amount of that ion, the rate at which release occurs, the pH of the solution, and the resulting osteoblast stimulating response. This effect is observed with respect to the particles of bioactive glass themselves and also in the ionic solutions derived from the glass particles. Accordingly, in the uses described below, particles of bioactive glass can be used in place of or in addition to the solutions derived from the particles.

Solid Compositions

The aqueous solutions can be dried, for example by spray drying or by drying in vacuo, to provide an antibacterial composition. The compositions can be incorporated into other solutions used in cell culture or other tissue engineering applications, such as cell culture media.

There are many types of cell culture media, each of which are essentially isotonic with the cells to be cultured. These include Dulbecco's minimal essential media, Hank's balanced salt solution, and others. The compositions described herein can be added to any of these solutions to enhance osteoblast proliferation, differentiation and/or function in the cell culture media. The cell culture media including the compositions described herein are also useful for other cell types, including fibroblasts, chondroblasts and other cells with a phenotype similar to osteoblasts.

III. Formulations Including Bioactive Glass

The compositions can be in a variety of forms. These include, for example, solid, semi-solid and liquid dosage forms such as tablets, pills, powders, liquid solutions or suspensions, suppositories, and injectable and infusible solutions. The preferred form depends on the intended mode of administration and therapeutic application and can be selected by one skilled in the art. Modes of administration can include oral, parenteral, subcutaneous, intravenous, intralesional or topical administration, or direct injection into a bony defect or an adjacent tissue locus. In most cases, the pharmaceutical compositions will be administered in the vicinity of the treatment site in need of tissue regeneration or repair.

The compositions can, for example, be placed into sterile, isotonic formulations with or without co-factors which stimulate uptake or stability. Solutions including the ions at appropriate concentrations and/or ratios can be lyophilized, stored under refrigeration and reconstituted prior to administration with sterile Water-For-Injection (USP).

The compositions can include conventional pharmaceutically acceptable carriers well known in the art (see for example Remington's Pharmaceutical Sciences, 16th Edition, 1980, Mac Publishing Company). Such pharmaceutically acceptable carriers can include other medicinal agents, carriers, genetic carriers, adjuvants, excipients, etc., such as human serum albumin or plasma preparations. The compositions are preferably in the form of a unit dose and will usually be administered as a dose regimen that depends on the particular tissue treatment.

The pharmaceutical compositions can also be administered, for example, in microspheres, liposomes, other microparticulate delivery systems, polymers or sustained release formulations placed in, near, or otherwise in communication with affected tissues or the bloodstream bathing those tissues.

Liposomes containing the compositions described herein can be prepared by well-known methods (See, e.g., DE 3,218,121; Epstein et al., Proc. Natl. Acad. Sci. U.S.A., 82, pp. 3688-92 (1985); Hwang et al., Proc. Natl. Acad. Sci. U.S.A., 77, pp. 4030-34 (1980); U.S. Pat. Nos. 4,485,045 and 4,544,545). Ordinarily, the liposomes are of the small (about 200-800 Angstroms) unilamellar type in which the lipid content is greater than about 30 mol. % cholesterol. The proportion of cholesterol is selected to control the optimal rate of release.

Dosing of the compositions can be via a single dose, sequential dosing, or continuous release.

Other Therapeutic Agents

In addition to the osteoblast-stimulating bioactive glass and/or extracts thereof, the formulations can include other therapeutic agents such as antibiotics, antivirals, healing promotion agents, anti-inflammatory agents, immunosuppressants, growth factors, anti-metabolites, cell adhesion molecules (CAMs), bone morphogenic proteins (BMPs), vascularizing agents, anti-coagulants, and topical anesthetics/analgesics.

Suitable growth factors include platelet-derived growth factor (PDGF), vascular endothelial growth factor (VEGF), epidermal growth factor (EGF), basic fibroblast growth factor (FGF), insulin-like growth factors (IGF-I and IGF-II), endothelial derived growth supplement (EDGS), keratinocyte growth factor (KGF), osteogenin, skeletal growth factor (SGF), osteoblast-derived(BDGFs), retinoids, growth hormone (GH), bone morphogenic proteins (BMPs), tissue growth factor-beta (TGF-β), CBFA-1 and transferrin.

IV. Devices

Devices can be prepared which include the compositions described herein, for example, dispersed in an implantable or extracorporeal biocompatible carrier material that functions as a suitable delivery or support system for the composition. Suitable examples of sustained release carriers include semi-permeable polymer matrices in the form of shaped articles such as suppositories or capsules. Implantable or microcapsular sustained release matrices include polylactides (U.S. Pat. No. 3,773,319; EP 058 481), copolymers of L-glutamic acid and ethyl-L-glutamate (Sidman et al., Biopolymers, 22, pp. 547-56 (1985)); poly(2-hydroxyethyl-methacrylate) or ethylene vinyl acetate (Langer et al., J. Biomed. Mater. Res., 15, pp. 167-277 (1981); Langer, Chem. Tech., 12, pp. 98-105 (1982)).

In one embodiment, the carrier includes a biocompatible matrix made up of particles or porous materials. The pores are preferably of a dimension to permit progenitor cell migration and subsequent differentiation and proliferation. Various matrices known in the art can be employed (see, e.g., U.S. Pat. Nos. 4,975,526; 5,162,114; 5,171,574 and PCT WO 91/18558).

The matrix can be formed, for example, by close packing particulate material into a shape spanning the particular tissue or bone defect to be treated. Alternatively, a biocompatible, preferably biodegradable material can be structured to serve as a temporary scaffold and substrate for recruiting migratory progenitor cells, and as a base for their subsequent anchoring and proliferation.

Useful matrix materials include, for example, collagen; hydrogels; homopolymers or copolymers of glycolic acid, lactic acid, and butyric acid, including derivatives thereof; and ceramics, such as hydroxyapatite, tricalcium phosphate and other calcium phosphates.

The bioactive glass or bioactive glass extracts of the invention may be used with, incorporated into or encapsulated within matrix carrier materials, such as hydrogels, to enable the release of the ions from the glass or extract in a controlled fashion. This release of the ions preferably will be controlled over time and may be a sustained release formulation. Various therapeutic agents, as described above, can be adsorbed onto or dispersed within the carrier material, and will also be released over time at the implantation site as the matrix material is slowly absorbed.

Implantable prosthetic devices including the compositions described herein can also be prepared. Such prosthetic implant can be selected for a particular treatment by the skilled practitioner, and can include materials such as metals and/or ceramics. The compositions can be moldable or machinable.

Examples of prosthetic devices include hip devices, screws, rods, cages for spine fusion, stents, plates, sheets, pins, valves, sutures, tubes and the like.

In one embodiment, the composition is disposed as a coating on prosthetic implants. For example, a surface region that is implantable adjacent to a target tissue in a mammal, preferably, a human, can be coated. The coating is present in an amount sufficient to promote enhanced tissue growth into the surface of the implant. The amount of the composition sufficient to promote enhanced tissue growth can be determined empirically by those of skill in the art using appropriate bioassays. Preferably, animal studies are performed to optimize the concentration of the composition components before a similar prosthetic device is used in the human patient. Such prosthetic devices will be useful for repairing orthopedic defects, injuries or anomalies in the treated mammal.

In vivo integration of implantable prosthetic devices into target tissue can be performed, for example, by providing the composition on a surface of a prosthetic device, and implanting the device in a mammal at a locus where the target tissue and the surface of the prosthetic device are maintained at least partially in contact for a time sufficient to permit enhanced tissue growth between the target tissue and the device.

V. Methods for Using the Compositions

The compositions and devices disclosed herein will permit the physician to treat a variety of tissue injuries, tissue degenerative or disease conditions and disorders that can be ameliorated or remedied by localized, stimulated tissue regeneration or repair. For example, the compositions and devices of the invention may be used to treat osteoblast-related tissue degenerative conditions.

The devices can be used to induce local tissue formation from a progenitor cell in a mammal by implanting the device at a locus accessible to at least one progenitor cell of the mammal. The devices can be used alone or in combination with other therapies for tissue repair and regeneration.

The devices can also be implanted in or surrounding a joint for use in cartilage and soft tissue repair, or in or surrounding nervous system-associated tissue for use in neural regeneration and repair.

The tissue specificity of the particular composition will determine the cell types or tissues that will be amenable to such treatments and can be selected by one skilled in the art. The ability to enhance tissue regeneration by administering the compositions described herein is thus not believed to be limited to any particular cell-type or tissue. The compositions and methods disclosed herein can be practiced to enhance new tissue inductive functions as they are discovered in the future.

The compositions and devices will permit the physician to obtain predictable bone and/or cartilage formation. The compositions and devices can be used to treat more efficiently and/or effectively all of the injuries, anomalies and disorders that have been described in the prior art of osteogenic devices. These include, for example, forming local bone in fractures, non-union fractures, fusions and bony voids such as those created in tumor resections or those resulting from cysts; treating acquired and congenital craniofacial and other skeletal or dental anomalies (see e.g., Glowacki et al., Lancet, 1, pp. 959-63 (1981)); performing dental and periodontal reconstructions where lost bone replacement or bone augmentation is required such as in a jaw bone; and supplementing alveolar bone loss resulting from periodontal disease to delay or prevent tooth loss (see e.g., Sigurdsson et al., J. Periodontol., 66, pp. 511-21 (1995)).

In addition to the osteoblast-stimulating bioactive glass and/or extracts thereof, the devices can also include a matrix including allogeneic bone. Such devices can also be implanted at a site in need of bone replacement to accelerate allograft repair and incorporation in a mammal.

The devices can also be used in cartilage repair, for example, following joint injury or in osteoarthritis treatment. The ability to enhance cartilage-inducing activity by administering the compositions described herein can permit faster or more extensive tissue repair and replacement.

The compositions and devices described herein will be useful in treating certain congenital diseases and developmental abnormalities of cartilage, bone and other tissues. Developmental abnormalities of the bone can affect isolated or multiple regions of the skeleton or of a particular supportive or connective tissue type. These abnormalities often require complicated bone transplantation procedures and orthopedic devices. The tissue repair and regeneration required after such procedures can occur more quickly and completely using the bioactive glasses as described herein.

Examples of heritable conditions, including congenital bone diseases, for which use of the morphogenic compositions and devices described herein will be useful include osteogenesis imperfecta, the Hurler and Marfan syndromes, and several disorders of epiphyseal and metaphyseal growth centers such as is presented in hypophosphatasia, a deficiency in alkaline phosphatase enzymatic activity.

Inflammatory joint diseases can also benefit from the compositions and devices described herein. These include infectious, non-infectious, rheumatoid and psoriatic arthritis, bursitis, ulcerative colitis, regional enteritis, Whipple's disease, and ankylosing spondylitis (also called Marie Strumpell or Bechterew's disease); the so-called “collagen diseases” such as systemic lupus erythematosus (SLE), progressive systemic sclerosis (scleroderma), polymyositis (dermatomyositis), necrotizing vasculitides, Sjogren's syndrome (sicca syndrome), rheumatic fever, amyloidosis, thrombotic thrombocytopenic purpura and relapsing polychondritis. Heritable disorders of connective tissue include Marfan's syndrome, homocystinuria, Ehlers-Danlos syndrome, osteogenesis imperfecta, alkaptonuria, pseudoxanthoma elasticum, cutis laxa, Hurler's syndrome, and myositis ossificans progressiva.

In one embodiment, the compounds are used to fill voids, including voids created during medical procedures. For example, during a root canal operation, the hollowed-out tooth can be filled with a composition including bioactive glass. This will help prevent bacterial infection until the tooth is ultimately filled. Also, bioactive glass-containing compositions can be used to fill the pockets that can develop between the teeth and gums. The compositions can also be used to fill voids, for example those present in aneurysms, and those formed surgically, such as removal of a spleen, ovary, gall bladder, or tumor.

VI. Bioassays

The utility of the compositions at enhancing bone and/or tissue growth can be demonstrated using conventional bioassays. Examples of useful bioassays are described in U.S. Pat. No. 5,344,654 to Rueger et al.

Feline and Rabbit Models

The feline and rabbit as established large animal efficacy models for osteogenic device testing have been described in detail (See, for example, U.S. Pat. No. 5,354,557 Oppermann et al.).

In the feline model, a femoral osteotomy defect is surgically prepared. Without further intervention, the simulated fracture defect would consistently progress to non-union. The effects of osteogenic compositions and devices implanted into the created bone defects can be evaluated by the following study protocol.

Briefly, the procedure is as follows: Sixteen adult cats each weighing less than 10 lbs. undergo unilateral preparation of a 1 cm bone defect in the right femur through a lateral surgical approach. In other experiments, a 2 cm bone defect can be created. The femur is immediately internally fixed by lateral placement of an 8-hole plate to preserve the exact dimensions of the defect.

Three different types of materials can be implanted in the surgically created cat femoral defects: group I is a negative control group which undergoes the same plate fixation with implants of 4M guanidine-HCl-treated (inactivated) cat demineralized bone matrix powder (GuHCl-DBM) (360 mg); group II is a positive control group implanted with biologically active demineralized bone matrix powder (DBM) (360 mg); and groups III and IV undergo a procedure identical to groups I-II, with the addition of the compositions to be evaluated.

All animals are allowed to ambulate ad libitum within their cages post-operatively. All cats are injected with tetracycline (25 mg/kg subcutaneously (SQ) each week for four weeks) for bone labeling. All but four group III and four group IV animals are sacrificed four months after femoral osteotomy.

In vivo radiomorphometric studies are carried out immediately post-op at 4, 8, 12 and 16 weeks by taking a standardized X-ray of the lightly-anesthetized animal positioned in a cushioned X-ray jig designed to consistently produce a true anterio-posterior view of the femur and the osteotomy site. All X-rays are taken in exactly the same fashion and in exactly the same position on each animal. Bone repair is calculated as a function of mineralization by means of random point analysis. A final specimen radiographic study of the excised bone is taken in two planes after sacrifice.

At 16 weeks, the percentage of groups III and IV femurs that are united, and the average percent bone defect regeneration in groups I-IV are compared. The group I GuHCl-DMB negative-control implants should generally exhibit no bone growth at four weeks, less than 10% at eight and 12 weeks, and about 16% (+/−10%) at 16 weeks. The group II DMB positive-control implants should generally exhibit about 15-20% repair at four weeks, 35% at eight weeks, 50% (+/−10%) at 12 weeks and 70% (+/−12%) by 16 weeks.

Excised test and normal femurs can be immediately studied by bone densitometry, or wrapped in two layers of saline-soaked towels, placed into sealed plastic bags, and stored at −20° C. until further study. Bone repair strength, load-to-failure, and work-to-failure are tested by loading to failure on a specially designed steel 4-point bending jig attached to an Instron testing machine to quantitate bone strength, stiffness, energy absorbed and deformation to failure. The study of test femurs and normal femurs yields the bone strength (load) in pounds and work-to-failure in joules. Normal femurs exhibit a strength of 96 (+/−12) pounds.

Following biomechanical testing, the bones are immediately sliced into two longitudinal sections at the defect site, weighed, and the volume measured. One-half is fixed for standard calcified bone histomorphometrics with fluorescent stain incorporation evaluation, and one-half is fixed for decalcified hemotoxylin/eosin stain histology preparation.

Selected specimens from the bone repair site are homogenized in cold 0.15 M NaCl, 3 mM NaHCO₃, pH 9.0 by a Spex freezer mill. The alkaline phosphatase activity of the supernatant and total calcium content of the acid soluble fraction of sediment are then determined.

Rabbit Model Bioassay for Bone Repair

This assay is described in detail in U.S. Pat. No. 5,354,557 to Oppermann et al. and Cook et al., J. Bone and Joint Surgery, 76-A, pp. 827-38 (1994). Ulnar non-union defects of 1.5 cm are created in mature (less than 10 lbs) New Zealand White rabbits with epiphyseal closure documented by X-ray. The experiment can include implantation of devices into at least eight rabbits per group as follows: group I negative control implants of 4M guanidine-HCl-treated (inactivated) demineralized bone matrix powder (GuHCl-DBM); group II positive control implants with biologically active demineralized bone matrix powder (DBM); group III implants with osteogenic protein alone; group IV implants with osteogenic protein/MPSF (morphogenic protein stimulatory factor) combinations, and group V controls receiving no implant. Ulnae defects are followed for the full course of the eight week study in each group of rabbits.

In another experiment, the marrow cavity of the 1.5 cm ulnar defect is packed with activated osteogenic protein in rabbit bone powder in the presence or absence of a MPSF. The bones are allografted in an intercalary fashion. Negative control ulnae are not healed by eight weeks and reveal the classic “ivory” appearance.

Tendon/Ligament-Like Tissue Formation bioassay

A modified version of the Sampath and Reddi rat ectopic implant assay (see above) is disclosed in PCT WO 95/16035. The modified assay monitors tendon and ligament-like tissue formation. This tendon/ligament-like tissue assay can be used to identify compositions that stimulate tendon/ligament-like tissue formation in a particular treatment site. The assay can also be used to optimize concentrations and treatment schedules for therapeutic tissue repair regimens.

It should be understood that the above experimental procedure can be modified within the skill of the art in a number of ways to be useful in determining whether a device is capable of inducing tendon and/or ligament-like tissue in vivo. It can be used to test various ion concentrations and/or ratios, and to produce an in vivo dose response curve useful in determining effective relative concentrations and/or ratios of ions in the bioactive glasses or extracts thereof.

Histological Evaluation

Histological sectioning and staining is preferred to determine the extent of osteogenesis in implants. Implants are fixed in Bouins Solution, embedded in paraffin, and cut into 6-8 μm sections. Staining with toluidine blue or hemotoxylin/eosin demonstrates clearly the ultimate development of endochondral bone. Twelve-day implants are usually sufficient to determine whether the implants contain newly-induced bone.

Biological Markers

Alkaline phosphatase (AP)activity can be used as a marker for osteogenesis. The enzyme activity can be determined spectrophotometrically after homogenization of the implant. The activity peaks at 9-10 days in vivo and thereafter slowly declines. Implants showing no bone development by histology have little or no alkaline phosphatase activity under these assay conditions. The assay is useful for quantification and obtaining an estimate of bone formation quickly after the implants are removed from the rat. Alternatively, the amount of bone formation can be determined by measuring the calcium content of the implant.

Gene expression patterns that correlate with endochondral bone or other types of tissue formation can also be monitored by quantitating mRNA levels using procedures known to those of skill in the art such as Northern Blot analysis. Such developmental gene expression markers can be used to determine progression through tissue differentiation pathways after osteogenic protein/MPSF treatments. These markers include osteoblastic-related matrix proteins such as procollagen α₂(I), procollagen α₁ (I), procollagen α₁ (III), osteonectin, osteopontin, biglycan, and alkaline phosphatase for bone regeneration (see e.g., Suva et al., J. Bone Miner. Res., 8, pp. 379-88 (1993); Benayahu et al., J. Cell. Biochem., 56, pp. 62-73 (1994)).

The procedures described above can be used to assess the ability of one or more of the compositions described herein to enhance bone and/or cartilage regeneration and repair in vivo. It is anticipated that the efficacy of any of the compositions described herein can be characterized using these assays. Various compositions, dose-response curves, naturally-derived or synthetic matrices, and any other desired variations on the device components can be tested using the procedures essentially as described.

The following are examples which illustrate the compositions and devices described herein, and methods used to characterize them. These examples should not be construed as limiting; the examples are included for purposes of illustration and the present invention is limited only by the claims.

### EXAMPLE 1

The Effect of the Ionic Dissolution Products of Bioglass D 45S5 on Human Primary Osteoblasts

The use of biomaterial resorption as a means to deliver morphogenic stimuli in cells and tissues was evaluated. Specifically, the effect of the ionic dissolution products of Bioglass D 45S5 on human primary osteoblasts in vitro was evaluated. Bioglass 45S5 is a bioactive glass ceramic material which resorbs initially by selective leaching of at least silicon, calcium and phosphorus ions followed by network dissolution mediated by surface re-polymerization.

The ionic dissolution products of Bioglass 45S5 stimulate gene transcription in human primary osteoblasts, as demonstrated using cDNA micro-array and real time PCR methodologies. The ionic dissolution products of Bioglass 45S5 can increase IGF-II availability in cells and tissues in two ways: i) by inducing the transcription of the growth factor and its carrier protein and ii) by regulating the dissociation of this factor from its binding protein resulting in an increase of free-active IGF-11, as determined by EIA. Free IGF-II increases the cell proliferation observed in cultures stimulated with the ionic dissolution products of Bioglass 45S5. The data demonstrate that the biomaterials described herein are useful not only for structural support, but also, through their resorption, for stimulating the intrinsic cellular pathways for bone growth, repair and regeneration.

Materials and Methods

Cell Culture and Stimulation.

Osteoblasts were isolated from trabecular bone of femoral heads taken during total hip arthroplasty using the method described by Beresford et al (Beresford et al., Metab. Bone Dis. and Rel. Res., 5:229-234 (1984)). Cultures were grown in DMEM (Dulbecco's modified Eagle's medium) supplemented with 10% fetal bovine serum (FBS), 2 mM L-glutamine, 50 U/ml penicillin G, 50 μg/ml streptomycin B and 0.3 μg/ml amphotericin B (complete medium) at 37° C., in 95% air humidity and 5% CO₂.

A solution containing the ionic dissolution products of Bioglass 45S5 was prepared by incubating 1 g of Bioglass 45S5 particulate (710-300 μm in diameter, U.S. Biomaterials Corp, USA) in 100 ml DMEM for 24 hours at 37° C. The particulates were removed by filtration through a 0.20 μm filter (Sartorius, UK) and the collected medium was supplemented as described above for the complete medium. The elemental content of this solution in calcium (Ca), silicon (Si), phosphorus (P) and sodium (Na) ions was determined by ICP analysis.

Human primary osteoblast cells at passages 2-3 were used. Cultures at approximately 75% confluence were stimulated with the ionic dissolution products of Bioglass. Non-stimulated cells were cultured in complete DMEM. After 48 hours the cells were released by trypsin, centrifuged and snap frozen in liquid N₂.

RNA Extraction

Total RNA was extracted using a phenol:chloroform method (Clontech Laboratories, Inc., Palo Alto, USA), and precipitated with isopropanol by 15000 g centrifugation at 4° C. The RNA pellet was washed with 80% ethanol, re-suspended in diethylpyrocarbonate-treated water. To remove genomic DNA, the RNA samples were then treated with DNase (0.10 units/μl of DNase 1, in DNAse I buffer, Clontech Laboratories, Inc., Palo Alto, USA). The concentration and purity of total RNA in each sample was determined by light absorbance at 260 nm and RNA integrity was assessed by electrophoresis on a denaturing agarose/formaldehyde/EtBr gel to verify that the RNA was intact.

Analysis of Gene Expression Using cDNA Microarrays

Gene expression analysis in four different donor primary osteoblast cell lines was performed using the ATLAS Platform (Atlas 1.2 Human array, Clontech Laboratories, Inc., Palo Alto, USA) which allows the simultaneous screening of 1172 genes. Briefly, gene specific primers were used for cDNA synthesis using Superscript II RNase H-(Life Technologies, UK) in the presence of [³²-P]-dATP (Amersham, UK). Labeled cDNA was purified from unincorporated nucleotides by gel filtration using CHROMA SPIN-200 columns. The incorporation of ³²P in the probe was determined by scintillation counting. Each filter was hybridized with equal amount of radioactive probe. Prehybridization and hybridization was done at 68° C. for 30 minutes and 16 hours respectively. Membranes were washed according to the manufacturers protocol. Arrays were scanned using a Molecular Dynamics 445 Sl PhosphorImager. Data analysis was performed using the Atlasimage 1.1 software package (Clontech Laboratories, Inc., Palo Alto, USA). Differential gene expression between stimulated and un-stimulated cells was normalized towards the expression of the ‘housekeeping genes’ 40S Ribosomal Protein S9 and 23 KDa highly basic protein.

Verification of c-DNA Microarray Data with Real Time Quantitative PCR

IGF-II that has been identified by the microarray analysis was selected for further analysis. RT reactions were carried out for each RNA sample using the Thermoscript RT-PCR System (Life Technologies, UK), according to manufacturer's protocol. Each reaction tube contained 1 μg of DNAse free total RNA in a total volume of 20 μl containing 1× cDNA Synthesis Buffer, 5 mM DTT, 40 U RNASEOUT, 1 mM dNTP Mix, 15U THERMOSCRIPT RT and 2.5 μM oligo (dT)₁₂₋₁₈ primer. RT reaction was carried out at 50° C. for 60 min and terminated by incubating at 85° C. for 5 min. Finally 2U of RNase H was added to each reaction and the reaction mixture was incubated for a further 20 min. at 37° C.

PCR primers and TaqMan probes for IGF-II were designed using Primer Express 1.0 Software program (PE Biosystems, UK). The human IGF-II cDNA sequence was obtained from GenBank (accession number S77035). The following forward and reverse primers were used 5′-GTGCTACCCCCGCCAAGT-3′ (located on exon four, anneals between residues 584 and 601) and 5′-CTGCTTCCAGGTGTCATATTGGA-3′ (located on exon 5, anneals between residues 696 and 674). The TaqMan probe sequence was 5-CTCCGACCGTGCTTCCGGACAACT-3′ (spans exon 4-exon 5 boundary, anneals between residues 623 and 646) and was labeled with the reporter fluorescent dye FAM (6-carboxyfluorescein), at the 5′ end and the fluorescent dye quencher TAMRA (6-carboxy-tetramethyl-rhodamine) at the 3′ end.

0.5 μl of each reaction mixture was subjected to PCR in a total volume of 25 μl containing 1× TaqMan Universal Master Mix (PE Biosystems, UK), 300 nM forward primer, 300 nM reverse primer and 50 nM probe, TaqMan Ix 18s ribosomal RNA endogenous control reagent (VIC fluorescent labeled probe and appropriate primers) was added in each reaction tube and served as internal amplification control. Each sample was run in quadruplicate. DNA amplification was carried out on the PE-ABI 7700 sequence detection system for the test samples, standards and no template controls using the sequence detector V 1.6 program. Cycling parameters, were: 50° C. for 5min, 95° C. for 10 min followed by 40 cycles of a two-stage temperature profile of 95° C. for 15s and 60° C. for 1 min. Data points collected following primer extension were analyzed at the end of thermal cycling. A threshold value was determined as 10 S.D. above the mean of the background fluorescence emission for all wells between cycles 1 and 15. The cycle number at which the fluorescence signal from a positive sample crosses this threshold was recorded.

Normalization of Data

Serial dilutions of human primary osteoblast cDNA were analyzed for each target, IGF-II and 18S, and threshold Cycle (C_{T} were plotted versus the log of the initial amount of cDNA to give a standard curve. C_{T}s for IGF-II and 18S RNA were adjusted using the appropriate standard curves. Then IGF-II adjusted C_{T} was normalized to 18S adjusted C_{T} to minimize variability in the results due to differences in the RT efficiency and RNA integrity among test samples.

Free IGF-II Elisa.

Cells were plated on a 24 well plate at a seeding density of 50000 cell/well and allowed to attach. Seven different donor osteoblast cell lines were used in the experiment (n=7). Two days following seeding cells were stimulated with ionic dissolution products of Bioglass® 45S5 and control medium, which were not supplemented with FCS. Free IGF-II was assayed in the supernatant of stimulated and non-stimulated cells after two days in culture using an IGF-II ELISA Kit (Diagnostic Systems Laboratories, Inc, Webster, USA) following the manufacturer's protocol. All samples were assayed in duplicate and free IGF-II levels were referred to total protein concentration. Protein concentration of the cell lysates was assayed by the Bradford dye binding method using bovine serum albumin as a standard (Bradford et al., Anal. Biochem., 72:248-254 (1976)).

Evaluation of Cell Proliferation

Cells were plated on a 24 well plate at a seeding density of 50000 cell/well and allowed to attach. Five different donor primary osteoblast cell lines were used in the experiment (n=5). Two days following seeding cells were stimulated with ionic dissolution products of Bioglass® 45S5 and control medium. After four days in culture the cells were released by trypsin and counted using a hemocytometer.

### Results

ICP Analysis

Analysis of the ionic composition of the two solutions used by ICP revealed an increase in concentration of Ca and most notably Si in the DMEM solution containing the ionic dissolution products of Bioglass 45S5, relative to control. These ions, along with P and Na, are constitutive elements of Bioglass 45S5 and their reaction kinetics in physiological solutions are well characterized chemically. However, their biological properties have not been described.

Gene Profiling using cDNA Microarrays

Microarray analysis of gene expression on four different donor cell lines revealed a similar pattern of gene expression. Approximately 5-7% of genes represented on the Atlas human 1.2 arrays were differentially expressed. These included insulin like growth factor II, and its binding protein IGFBP-3. Gene transcription of both these molecules was induced. Also induced were proteases (MMP-2 and cathepsin-D) that have been shown to cleave IGF-II from their binding proteins and release the active form of the molecule. MMP- 14, a previously non-described IGFBP cleaving protease, shows a similar pattern of induction suggesting possible involvement in the process. Steady state mRNA transcripts for the IGF-II receptor was relatively unaffected by the stimulus. The analysis identified 60 mRNA species that were upregulated greater than twofold in the treated cultures compared to the untreated control (Table 1). Only five genes were identified as down-regulated, including E-16 amino acid transporter, c-jun terminal kinase 2, polycystin precursor, Sp2 protein and proteasome inhibitor HP131 subunit.

TABLE I
List of Genes Up-Regulated or Down-Regulated Greater
Than Twofold in Human Osteoblasts Treated with the
Ionic Products of Bioactive Glass Dissolution

| GeneBank | | | |
|---|---|---|---|
| Accession | | | |
| No. | Protein/Gene | Ratio | Function |
| M59040 | CD44 antigen hematopoietic | 7 | Cell surface receptor |
| | form precursor | | |
| U12779 | MAP kinase-activated protein | 6 | Signal transduction |
| | kinase 2 (MAPKAP kinase 2) | | |
| X06256 | Integrin beta 1; fibronectin | 6 | Cell surface receptor |
| | receptor beta subunit | | |
| AF040105 | RCL growth-related | 5 | Growth related gene |
| | c-myc-responsive gene | | |
| D15057 | Defender against cell death 1, | 4.5 | Apoptosis |
| | (DAD-1) | | |
| Y00371 | Heat shock cognate 71-kDa | 4.5 | Heat shock protein |
| | protein | | |
| X04106 | Calpain; calcium-dependent | 4.1 | Apoptosis |
| | protease small subunit | | |
| X59798 | G1/S-specific cyclin D1 | 4 | Cell cycle regulator |
| D11428 | Peripheral myelin protein 22 | 4 | Cell surface antigen |
| M34079 | 26S protease regulatory | 4 | Transcription factor |
| | subunit 6A; TAT-binding | | |
| | protein 1 | | |
| D26512 | Matrix metalloproteinase | 3.6 | Matrix component |
| | 14 precursor (MMP14) | | |
| J03075 | Protein kinase C substrate | 3.5 | Signal transduction |
| | 80-kDa protein heavy chain | | |
| U09579 | Cyclin-dependent kinase | 3.4 | Cell cycle regulator |
| | inhibitor 1 (CDKN1A) | | |
| L19185 | Natural killer cell enhancing | 3.3 | Antioxidant |
| | factor (NKEFB) | | |
| M2964S | Insulin-like growth factor II | 3.2 | Growth factor |
| | (IGF2) | | |
| L11285 | Dual specificity mitogen- | 3 | Signal transduction |
| | activated protein kinase kinase 2 | | |
| M13194 | DNA excision repair protein | 3 | DNA repair |
| | ERCC1 | | |
| U07418 | MutL protein homolog | 3 | DNA repair |
| X69391 | 60S ribosomal protein L6 | 3 | Transcription |
| M13667 | Major prion protein precursor | 3 | Cell surface antigen |
| M37722 | N-sam; fibroblast growth factor | 3 | Cell surface receptor |
| | receptor 1 precursor | | |
| X79389 | Glutathione S-transferase T1 | 3 | Enzyme |
| L42379 | Bone-derived growth | 3 | Growth factor |
| | factor 1 (BPGF1) | | |
| K00065 | Cytosolic superoxide | 3 | Enzyme |
| | dismutase 1 (SOD1) | | |
| M26880 | Ubiquitin | 2.9 | Enzyme |
| M17733 | Thymosin beta 4; FX | 2.9 | Nuclear protein |
| J03210 | Matrix metalloproteinase 2 | 2.7 | Matrix Component |
| | (MMP2) | | |
| M37435 | Macrophage-specific colony | 2.6 | Growth factor |
| | stimulating factor (MCSF) | | |
| M92843 | Tristetraproline | 2.5 | Transcription factor |
| D90209 | cAMP-dependent transcription | 2.3 | Transcription factor |
| | factor ATP-4 | | |
| X69550 | rho GDP dissociation-inhibitor 1 | 2.3 | Signal transduction |
| M23619 | High mobility group protein | 2.3 | Nuclear protein |
| | (HMG-I) | | |
| X15480 | Glutathione S-transferase pi | 2.3 | Enzyme |
| X03124 | Metalloproteinase inhibitor | 2.2 | Matrix component |
| | 1 precursor (TIMP1) | | |
| M14219 | Decorin; bone proteoglycan II | 2.2 | Matrix component |
| | precursor | | |
| J05594 | TIMP-2 | 2.1 | Matrix component |
| M11233 | Cathepsin D precursor | 2 | Enzyme |
| X60188 | Extracellular signal-regulated | 2 | Signal transduction |
| | kinase 1 (ERK1) | | |
| M77234 | fte-1 | 2 | Transcription factor |
| AF060515 | Cyclin K | 2 | Cell cycle regulator |
| M36340 | ADP-ribosylation factor 1 | 2 | Signal transduction |
| L35253 | Mitogen-activated protein | 2 | Signal transduction |
| | kinase p38 (MAP kinase p38) | | |
| M36429 | Guanine nucleotide-binding | 2 | signal transduction |
| | protein -G-i/G-s/G-t beta | | |
| | subunit 2 | | |
| U32944 | Cytoplasmic dynein light chain 1 | 2 | Translocation |
| L07541 | Replication factor C 38-kDa | 2 | DNA synthesis |
| | subunit | | |
| J00123 | Proenkephalin A precursor | 2 | Cell surface receptor |
| M65212 | Membrane-bound & soluble | 2 | Enzyme |
| | catechol-O-methyltransferase | | |
| U04847 | Inil | 2 | Transcription factor |
| L31881 | Nuclear factor 1 (NP1) | 2 | Transcription factor |
| M30257 | Vascular cell adhesion protein | 2 | Cell adhesion |
| | 1 precursor (V-CAM 1) | | |
| D28468 | DNA-binding protein | 2 | Transcription factor |
| | TAXREB302 | | |
| M62831 | Transcription factor ETR101 | 2 | Transcription factor |
| J03746 | Microsomal glutathione | 2 | Enzyme |
| | S-transferase 12 | | |
| X06985 | Herne oxygenase 1 (HO1) | 2 | Enzyme |
| M35977 | Vascular endothelial growth | 2 | Growth factor |
| | factor precursor (VEGF) | | |
| M36717 | Ribonuclease/angiogenin | 2 | Nuclear protein |
| | inhibitor (RAI) | | |
| D16431 | Hepatoma-derived growth factor | 2 | Growth factor |
| | (HDGF) | | |
| K03515 | Neuroleukin (NLK) | 2 | Enzyme |
| M24545 | Monocyte chemotactic protein | 2 | Cytokine |
| | 1 precursor (MCP1) | | |
| X04602 | Interleukin-6 precursor (1L-6) | 2 | Cytokine |
| AF077866 | E16 amino acid transporter | .5 | Transporter |
| L31951 | c-jun N-terminal kinase 2 | .5 | Signal transduction |
| | (JNK2) | | |
| U24497 | Polycystin precursor | .5 | Cell adhesion |
| M97190 | Sp2 protein | .5 | Transcription factor |
| D88378 | Proteasome inhibitor | .4 | Enzyme |
| | HPI31 subunit | | |

Corroboration of Results with Taqman Real Time PCR

Taqman real time PCR was used to confirm induction of IGF-II mRNA expression demonstrated by cDNA microarray analysis. Expression and induction of IGF-II followed the same pattern in all four donor osteoblast cell lines examined.

Free IGF-II ELISA

Free IGF-II represents the fraction of the molecule, which is not bound to IGF binding proteins (IGFBPs) and hence represents the active form of IGFII. The ionic dissolution products of Bioglass 45S5 were shown to statistically increase the concentration of free IGF-II by approximately 70%.

Evaluation of Cell Proliferation

Osteoblast proliferation was increased 50.2% (P<0.001) over control, following four days of stimulation with the ionic dissolution products of Bioglass 45S5. The stimulatory effect on cell proliferation observed is believed to be mediated by IGF-II, which has been described as a potent mitogenic, growth factor for osteoblasts.

Effects of Stimulation of Cells by Ionic Dissolution Products

Chemical substances released by the bioactive glass substrate are believed to account for the observed changes in cellular performance. Bioglass 45S5 resorbs initially by selective leaching of Si, Ca, and P ions followed by network dissolution mediated by surface re-polymerization.

Using cDNA microarray methodology, the data show that human primary osteoblast transcription is directly regulated by the ionic dissolution products of Bioglass 45S5. Among the genes which were found to be up-regulated in human primary osteoblasts were IGF-II and to a lesser extent its carrier protein IGFBP-3.

IGF-II is an anabolic peptide of the insulin family and constitutes the most abundant growth factor in bone (Mohan et al., 1988, Bautista et al., 1990). It is produced locally by bone cells and is considered to exert mostly paracrine or autocrine effects. Nonetheless, differences in IGF-II expression occur and can significantly impact bone cell function in various physiological and pathological conditions. In vitro studies using osteoblasts of various animal sources have shown that IGF-II is a potent inducer of osteoblast proliferation and collagen synthesis.

The majority of IGF-II in vivo is found bound to IGE binding proteins (IGFBPs). The latter can inhibit or potentiate its biological activity, form storage complexes with IGFs or stabilize IGFs in the circulation for slow release into the peripheral tissues. Therefore IGF-II activity appears to be influenced not only by the level of expression of IGF-II polypeptide but also by the type and concentrations of IGFBPs present locally. Thus changes in IGFBPs expression by bone cells can well contribute to the effectiveness of IGF-II in the tissue.

The induction of IGF-II m-RNA expression represents a true difference in IGF-II protein synthesis and IGF-II availability. IGF-Il bioavailability at the local level is regulated through IGFBPs limited proteolysis by several proteases resulting in IGF-II release in its free ‘active’ form. These include members of the metalloproteinase family, such as MMP I and 2 and cathepsin-D (Conover et al., 1994), some of which were found to be transcriptionally induced in the system described in this example. This effect was correlated with a statistically significant increase of free-active IGF-II in cells stimulated with the ionic dissolution products of Bioglass 45S5.

The ionic dissolution products of Bioglass 45S5 can increase the availability of IGF-II in cells and tissues in two ways, (i) by inducing the transcription of the growth factor and its carrier protein and (ii) by regulating the dissociation of this factor from its binding protein. One of the direct effects of free IGF-II is the observed increase in cell proliferation.

In summary, the ionic dissolution products of Bioglass 45S5 induce the bioavailability of IGF-II, IGFBP3, MMP2, MMP14, TIMP1, TIMP2, procollagen a2, Decorin, c-jun, c-myc, calcium proteinase (calpain) and DAD 1 in human primary osteoblasts, and effect bone cell proliferation and differentiation as well as bone tissue growth.

Moreover, the ionic dissolution products of Bioglass were found to upregulate genes, at a rate greater than twofold in human osteoblasts, such as CD44 antigen hemotopoietic form precursor, MAP kinase-activated protein kinase 2, integrin beta 1, RCL growth-related c-myc-responsive gene, defender against cell death 1 (DAD- 1), cyclin D1, MMP14, CDKN1A, IGF-II, MMP2, TIMP1, decorin, TIMP-2, extracellular signal-regulated kinase 1, cyclin K, ADP-ribosylation factor 1, MAP kinase p38, nuclear factor 1 (NFI), vascular endothelial growth factor precursor (VEGF), among others. It is believed that the upregulation of these genes by bioactive glass or glass extracts as taught herein contributes, directly or indirectly, to the stimulation of osteoblast proliferation, differentiation and /or function.

While the invention has been described in detail and with reference to specific embodiments thereof, it will be apparent to one skilled in the art that various changes and modifications can be made without departing from the spirit and scope thereof.

What is claimed is:

1. A method for enhancing osteoblast production comprising exposing osteoblasts to a composition comprising an effective amount of bioactive glass for stimulation of osteoblast proliferation, differentiation, fimction, or a combination thereof.

2. The method of claim 1, wherein the bioactive glass comprises by approximate weight percentage:

| Component | Percent |
|---|---|
| SiO₂ | 42-52 |
| CaO | 15-25 |
| Na₂O | 15-25 |
| P₂O₅ | 1-9 |

and wherein the bioactive glass is in the form of matrices for cell culture, sols, gels, particles, or fibers.

3. The method of claim 2, wherein the bioactive glass is in the form of non-interlinked particles of bioactive glass.

4. The method of claim 1, wherein the composition further comprises one or more therapeutic agents.

5. The method of claim 4, wherein therapeutic agent(s) are selected from the group consisting of healing promotion agents, growth factors, anti-inflammatory agents, and topical anesthetics.

6. The method of claim 1, wherein the bioactive glass comprises by approximate weight percentage:

| Component | Percent |
|---|---|
| SiO₂ | 45 |
| CaO | 24.5 |
| Na₂O | 24.5 |
| P₂O₅ | 6. |

7. The method of claim 3, wherein the size range of the particles is less than about 1200 microns as measured by SEM or laser light scattering techniques.

8. The method of claim 3, wherein the size range of the particles is about 100 to about 800 microns as measured by SEM or laser light scattering techniques.

9. The method of claim 3, wherein the size range of the particles is less than about 90 microns as measured by SEM or laser light scattering techniques.

10. The method of claim 1 wherein the composition is used in a device selected from the group consisting of prosthetic implants, sutures, stents, screws, plates, valves and tubes.

11. A method for stimulating osteoblast proliferation, differentiation, function, or a combination thereof comprising exposing osteoblasts to an effective amount of a bioactive glass extract composition.

12. The method of claim 11 wherein the bioactive glass extract composition comprises an aqueous solution comprising about 1 to about 100 ppm Si, about 10 to about 150 ppm Ca and about 5 to about 50 ppm P.

13. The method of claim 11 wherein the bioactive glass extract composition is incorporated into a matrix carrier material to provide controlled release of the extract composition.

14. The method of claim 13 wherein the matrix carrier material is a hydrogel.

15. The method of claim 11 wherein the bioactive glass extract composition is dispersed in an implantable or extracorporeal biocompatible carrier material.

16. A method for inducing local tissue formation from a progenitor cell in a mammal, comprising exposing osteoblasts to an effective amount of a bioactive glass or bioactive glass extract composition.

17. A method for accelerating allograft repair in a mammal, comprising contacting an allograft with an effective amount of a bioactive glass or bioactive glass extract composition.

18. A method for promoting in vivo integration of an implantable prosthetic device to enhance the bond strength between the prosthesis and the existing target tissue at the joining site, comprising exposing osteoblasts to an effective amount of a bioactive glass or bioactive glass extract composition.

19. A method for treating osteoblast-related tissue degenerative conditions in a mammal, comprising administering to the mammal an effective amount of a bioactive glass or bioactive glass extract composition.

20. A composition comprising an extract of bioactive glass, wherein the bioactive glass has a composition by approximate weight percentage:

| Component | Percent |
|---|---|
| SiO₂ | 42-52 |
| CaO | 15-25 |
| Na₂O | 15-25 |
| P₂O₅ | 1-9 |

and wherein the extract of bioactive glass stimulates osteoblast proliferation, differentiation, function or a combination thereof.

21. The composition of claim 20, wherein the bioactive glass has a composition by approximate weight percentage:

| Component | Percent |
|---|---|
| SiO₂ | 45 |
| CaO | 24.5 |
| Na₂O | 24.5 |
| P₂O₅ | 6. |

22. The composition of claim 20 wherein the extract of bioactive glass comprises an aqueous solution comprising about 1 to about 100 ppm Si, about 10 to about 150 ppm Ca and about 5 to about 50 ppm P.

23. The composition of claim 22 wherein the extract of bioactive glass comprises an aqueous solution comprising about 3 to about 30 ppm Si, about 60 to about 100 ppm Ca and about 10 to about 40 ppm P.

24. A method for stimulating osteoblast production comprising:
exposing osteoblasts to an effective amount of bioactive glass or bioactive glass extract; and
thereby upregulating one or more genes involved in osteoblast proliferation, differentiation, function or a combination thereof.

25. The method of claim 24 wherein the one or more genes are selected from the group consisting of CD44, MAP kinase activated protein kinase 2, integrin β 1 and RCL growth-related c-myc responsive gene.

26. The method of claim 24, wherein the one or more genes are selected from the group consisting of IGF-II, IGFBP3, MMP2, MMP14, TIMP1, TIMP2, procollagen a2, decorin, c-jun, c-myc, calpain, and DAD 1.

27. The method of claim 26, wherein the one or more genes are selected from the group consisting of IGF-II, MMP2, MMP14, TIMP 1, calpain and DAD 1.

28. The method of claim 24 wherein the bioactive glass extract comprises an aqueous solution comprising about 1 to about 100 ppm Si, about 10 to about 150 ppm Ca and about 5 to about 50 ppm P.

29. A method for upregulating one or more genes involved in the proliferation, differentiation and/or function of osteoblasts comprising exposing osteoblasts to an effective amount of bioactive glass.

30. The method of claim 29 wherein the one or more genes are selected from the group consisting of CD44, MAP kinase activated protein kinase 2, integrin β 1 and RCL growth-related c-myc responsive gene.

31. The method of claim 29, wherein the one or more genes are selected from the group consisting of IGF-II, IGFBP3, MMP2, MMP14, TIMP1, TIMP2, procollagen a2, decorin, c-jun, c-myc, calpain, and DAD 1.

32. The method of claim 31, wherein the one or more genes are selected from the group consisting of IGF-II, MMP2, MMP14, TIMP 1, calpain and DAD 1.

33. The method of claim 29 wherein the bioactive glass extract comprises an aqueous solution comprising about 1 to about 100 ppm Si, about 10 to about 150 ppm Ca and about 5 to about 50 ppm P.

34. A method for increasing IGF-II availability in cells and tissues comprising exposing the cells and tissues to an effective amount of a bioactive glass extract comprising about 1 to about 100 ppm Si, about 10 to about 150 ppm Ca and about 5 to about 50 ppm P.
